(19) United States
(12) Patent Application Publication
(10) Pub. No.: US 2024/0398569 A1
(43) Pub. Date: Dec. 5, 2024

(54) Annuloplasty Ring Sizer

(71) Applicant: Northwestern University, Evanston, IL (US)

(72) Inventor: Patrick M. McCarthy, Chicago, IL (US)

(73) Assignee: Northwestern University, Evanston, IL (US)

(21) Appl. No.: 18/805,265

(22) Filed: Aug. 14, 2024

Related U.S. Application Data
(63) Continuation of application No. 17/251,728, filed on Dec. 11, 2020, now Pat. No. 12,083,015.
(60) Provisional application No. 62/683,397, filed on Jun. 11, 2018.

(51) Int. Cl. A61F 2/24; A61B 90/00

(52) U.S. Cl. CPC A61F 2/2496; A61B 90/06; A61F 2/2445; A61B 2090/061; A61F 2250/0097

(57) ABSTRACT

A ring sizer including a face plate that has markings usable to provide measurements of a valve. The sizer may include a clear face plate on which the markings are provided such that the valve is visible through the face plate facilitating accurate measurements of various valve dimensions and optimal ring sizing.

### RELATED APPLICATIONS

This application is a continuation of U.S. patent application Ser. No. 17/251,728 filed Dec. 11, 2020 entitled Annuloplasty Ring Sizer, which is a U.S. National Phase of and claims priority to International Patent Application No. PCT/US2019/036623, International Filing Date Jun. 11, 2019, entitled Annuloplasty Ring Sizer; which claims benefit of U.S. Provisional Application Ser. No. 62/683,397 filed Jun. 11, 2018 entitled Annuloplasty Ring Sizer; all of which are incorporated herein by reference in their entireties.

### BACKGROUND OF THE INVENTION

Annuloplasty procedures involve remodeling a heart valve by installing a ring around the valve annulus. The ring is sized to reduce the size of the enlarged valve back to an optimal size to reestablish coaptation of the valve leaflets. These rings come in a variety of shapes and sizes. As such, a key component of a successful annuloplasty procedure is an accurate measurement of the target valve so an optimally-sized annuloplasty ring can be selected. Measurement of the native valve is accomplished with a valve sizer.

Valve sizers are typically valve-shaped rings or similar structures attached to an elongated instrument handle. The sizers are provided in sets that include numerous sizers, identified by a single number indicating the diameter across the annulus in millimeters. However, valves, such as mitral valves, are not circular, and thus generalizing them using a single measurement can result in inaccuracies, due to the variety of valve shapes. Compounding this problem is the fact that the limited information provided by existing sizers is easily misinterpreted by physicians, as valve repair is not a common procedure.

If a ring is improperly selected, implantation of the ring will lead to a “failed” repair. For example, if the ring is too large, incomplete coaptation will occur, resulting in residual valve leakage. A ring that is too small can also result in residual leakage, as well as a condition known as “systolic anterior motion,” which is an obstruction of flow within the heart.

It would thus be advantageous to provide a valve sizer that provides more information to a physician, such that an optimal ring may be selected.

### OBJECTS AND SUMMARY OF THE INVENTION

In one aspect of the invention, a valve sizer is provided that allows a physician to take various measurements of a valve.

Another aspect of the invention involves a valve sizer that allows a physician to measure a valve without requiring the physician to insert numerous sizers on a trial-and-error basis.

Another aspect of the invention provides a sizer made of a clear material, such as plastic, that includes a scale, allowing the physician to see through the sizer such that he or she may measure the valve.

Another aspect of the invention is a sizer that is attached to a handle made of a material, such as any of a variety of metals or plastics, that may have various lengths or have an elongatable or telescoping configuration.

Yet another aspect of the invention is a sizer having a handle that is flexible, allowing easier access to the heart.

Still another aspect of the invention is a sizer that may be used as an aid in repair or replacement of any heart valve undergoing repair or replacement.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a is an elevation of an embodiment of a sizer of the invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

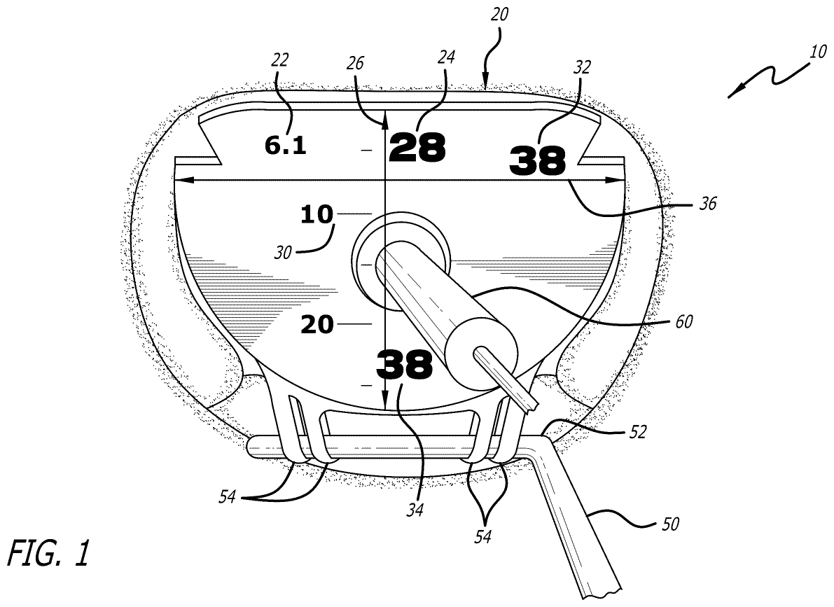

Referring now to FIG. 1 there is shown a sizer 10 of the invention. Sizer 10 generally includes a face plate 20 connected to a handle 50.

The face plate 20 may be formed of a variety of materials, such as metals or plastics. Advantageously, the face plate 20 could be formed of clear plastic, such that the extents of the native valve annulus may be visible through the face plate 20.

The face plate 20 includes a plurality of markings. Beginning in the upper left-hand corner of the face plate, as depicted in FIG. 1, is a first marking 22 denoting the surface area of the sizer. First marking 22 in FIG. 1 is 6.1 indicating that the surface area of the sizer is 6.1 cm².

Moving clockwise around the face plate 20, the second marking 24 denotes the anterior posterior (AP) distance. In FIG. 1, AP distance is 28 mm, as an example. As denoted by the vertical arrows 26 next to the marking 24, the AP distance is the distance from the top of the sizer to the bottom of the sizer in millimeters. To provide further clarification, the arrow 26 may be color-coordinated with the second marking 24.

Typically, AP distance is only available from charts, and is an estimation. The present invention provides actual AP measurement. In one embodiment, a calibrated scale 30 is provided next to the arrows 26 such that a physician can use the face plate 20 to obtain a measurement of the AP distance, if the sizer height does not match the valve height.

Continuing clock-wise, a third numeral marking 32 is provided, shown as the number 38 in the example of FIG. 1. This number denotes the maximum distance across the sizer 10, in millimeters. This is the sole number typically provided by sizers and is used to identify the sizer itself. For example, the sizer of FIG. 1 would typically be referred to as a “38 sizer.” Keeping with this convention, a fourth marking 34 is provided (as a 38 in FIG. 1), in a larger font at the bottom of the face plate 20.

To avoid confusion, an arrow 36 may be provided under the marking 32, and optionally color-coordinated with marking 32, that shows the third marking 32 corresponds to the maximum distance across the sizer 10.

The face plate 10 is connected to a handle 50. The handle 50 may be made of various metals, plastics, or similar materials, and may be flexible or rigid, depending on the surgical approach to the valve. Additionally, the handle 50 may be telescoping or otherwise capable of being elongated.

In FIG. 1, the handle 50 is shown as having a 90 degree bend 52 that is hingedly-connected to the face plate 20 with connectors 54 for easy translation through a vessel. The angular relationship between the face plate 20 and the handle 50 may be controlled with a push rod 60. The push rod 60 may be connected to the center of the face plate 20 via a thread or other connection mechanism. The push rod 60 is offset from the handle 50 to provide leverage and is preferably located in a location where it will not interfere with the visualization of the markings.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method of measuring a native valve for use in selecting an annuloplasty ring, the method comprising:
providing a clear face plate having a plurality of markings thereon against the native valve;
visualizing the native valve through the clear face plate; and
using the plurality of markings to measure extents of the native valve.

2. The method of claim 1, wherein using the plurality of markings to measure the extents of the native valve includes measuring a maximum anterior posterior dimension of the native valve.

3. The method of claim 2, wherein measuring the maximum anterior posterior dimension of the native valve includes using a calibrated scale located on the clear face plate.

4. The method of claim 3, wherein using the calibrated scale includes comparing the extents of the native valve to a plurality of linear horizontal lines each spaced vertically apart from one another.

5. The method of claim 4, wherein measuring the maximum anterior posterior dimension of the native valve includes comparing the extents of the native valve to a vertical line and a vertical linear dimension marking illustrating a maximum anterior posterior dimension of the clear face plate.

6. The method of claim 2, wherein using the plurality of markings to measure the extents of the native valve further includes measuring a maximum width of the native valve, and wherein the maximum width is a distance measured orthogonally to the maximum anterior posterior dimension.

7. The method of claim 6, wherein measuring the maximum width of the native valve includes comparing the extents of the native valve to a horizontal line and a horizontal linear dimension marking illustrating a maximum width of the clear face plate.

8. The method of claim 1, wherein the method includes aligning the clear face plate with the native valve by manipulating a handle to which the clear face plate is hingedly attached.

9. The method of claim 1, wherein the method includes controlling an angle defined between a handle attached to the clear face plate and a push rod attached to the face plate by manipulating the push rod.

10. A method of measuring a native valve for use in selecting an annuloplasty ring, the method comprising:
positioning a clear face plate against the native valve;
viewing extents of the native valve through the clear face plate; and
measuring the native valve by comparing the extents of the native valve to a vertical line and a linear vertical dimension marking illustrating a maximum height of the clear face plate, and a horizontal line intersecting the vertical line and located adjacent to a linear horizontal dimension marking illustrating a maximum width of the clear face plate.

11. The method of claim 10, wherein measuring the native valve further includes comparing the extents of the native valve to a calibrated scale configured for determining a maximum anterior posterior dimension of the native valve.

12. The method of claim 11, wherein comparing the extents of the native valve to the calibrated scale includes comparing the extents to a plurality of horizontal lines each spaced vertically apart from one another adjacent the vertical line.

13. The method of claim 12, wherein comparing the extents to the plurality of horizontal lines includes using a plurality of linear vertical dimension markings each numerically representing a linear vertical distance between one of the plurality of horizontal lines and an outer edge of the clear face plate.

14. A method of measuring a native valve for use in selecting an annuloplasty ring, the method comprising:
positioning a clear face plate on the native valve;
visualizing extents of the native valve through the clear face plate; and
comparing the extents of the native valve to calibrated scale to measure an anterior posterior dimension of the native valve and a horizontal line adjacent a linear horizontal dimension marking to measure a maximum width of the native valve.

15. The method of claim 14, wherein the method first comprises selecting the clear face plate based on a linear vertical dimension marking representing a maximum anterior posterior dimension of the clear face plate.

16. The method of claim 15, further comprising selecting the clear face plate based on a plurality of linear vertical dimension markings of the calibrated scale each numerically representing a distance between one of a plurality of horizontal lines and an outer edge of the clear face plate.

17. The method of claim 16, further comprising selecting the clear face plate based on the linear horizontal dimension marking.

18. The method of claim 17, further comprising selecting the clear face plate based on a numerical marking representing a surface area of the clear face plate.

19. The method of claim 14, wherein positioning the clear face plate on the native valve includes hingedly attaching the native valve to telescoping handle.

20. The method of claim 19, wherein positioning the clear face plate on the native valve includes manipulating a push rod attached to the clear face plate to change an angle defined between the clear face plate and the telescoping handle.
